(19) United States
(12) Patent Application Publication
(10) Pub. No.: US 2016/0346335 A1
(43) Pub. Date: Dec. 1, 2016

(54) Honey-based dressing for the treatment of wounds and burns

(71) Applicant: Carlos ALVARADO EGLI, Guatemala (GT)

(72) Inventor: Carlos A. Alvarado, Guatemala (GT)

(21) Appl. No.: 14/414,171

(22) PCT Filed: Feb. 5, 2013
(86) PCT No.: PCT/IB2013/050962

§ 371 Date: Sep. 16, 2015

(51) Int. Cl. A61K 35/644; A61L 15/44; A61L 15/28; A61K 36/48; A61K 31/095

(52) U.S. Cl. CPC A61K 35/644; A61K 36/48; A61K 31/095; A61L 15/28; A61L 15/44; A61L 2300/30; A61L 2300/412

(57) ABSTRACT

A practical active dressing (PAD) is disclosed that includes a honey-based composition which is to be applied to a patient's wound.

Additionally a method of manufacturing a practical active dressing is disclosed.

The formulation, with considerable wound and ulcer healing capacity and strong antimicrobial activity, incorporate the combination of active ingredients of ripe honey, gelling agents, plant extracts and chemical compounds.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### THE NAMES OF THE PARTIES TO A JOINT RESEARCH AGREEMENT

“Not Applicable”

### REFERENCE TO A “SEQUENCE LISTING

“Not Applicable”

### STATEMENT REGARDING PRIOR DISCLOSURES BY THE INVENTOR OR A JOINT INVENTOR

“Not Applicable”

### FIELD OF THE INVENTION

The invention generally relates to therapeutic compositions containing honey, gelling agents, plant extracts and chemical compounds. Processes of preparing and use the compositions of the present invention are also provided.

### BACKGROUND OF THE INVENTION

The use of honey in treating wounds have been long known, with such use being recorded in 4,000 year old Sumerian clay tablets and was one of the most cited ingredients in several topic wound preparations in Egyptian and Roman culture (Lindholm, 2003). The Edwin Smith papyrus (1500 b.C.) suggests protecting the wounds with fresh meat (haemostatic property) and application of butter and honeying (Dealey, 2003).

The past 2 decades have produced more advances in wound care than have the previous 2000 years as a result of rapid expansion in the knowledge of the healing process, and in this sense, the honey-derived product of the present invention comprise three properties that are essential for the repair of damaged tissues and assisting wound healing: A. Antibacterial Activity (by means of pH diminution) B. performing autolytic debridement and C. induction of angiogenesis.

### pH Reduction and Bacterial Control

In the PAD composition three products are incorporated for purposes of the pH reduction and bacterial control: raw honey, methyl-Phenol [Policresulen (Condensation product of metacresolsulfonic acid & methanal)], and acetic acid.

The value of honey as an antibacterial has since long been recognized. Hydrogen peroxide—already present (residual) and/or generated by glucose oxidase activity upon dilution of honey—as well as phenol constituents are considered major antibacterial factors (Molan, 2002), (Weston et. al, 1999).

Van Den, Berg in US Patent Application 20090304780 assessed the antioxidant and anti-inflammatory properties of several honeys [American buckwheat honey (Fagopyrum esculentum), Chilean honey (Ch), Active manuka (Leptospermum scoparium), HA, macadamia honey (Macadamia integrifolia); and HB, kiawe honey (Prosopis pallida)] using in vitro assays for testing their ability to inhibit the free radicals known as reactive oxygen species (ROS).

Most pronounced antioxidant activities, according to Van Den Berg, were found for American buckwheat honey from the state of New York with phenolic constituents present in relatively large amounts. Phenolic compounds may also exert antibacterial activity, whereas low pH and high free acid content may be other factors beneficial to healing of wounds.

NY buckwheat honey was found to have relatively strong acid properties, represented by low pH and high free acid content. Measurement of pH using a solution of NY buckwheat honey in water (25% w/v) showed pH 3.3, which is considered low in comparison with the average pH 3.9 for non-tropical honeys with a typical range of 3.4 to 6.1 (National Honey Board, 2005).

Compared to other types of honey, buckwheat honey is a rich source of phenolic antioxidants (Schramm et al, 2003). Since phenolic compounds also have antibacterial activity, (Weston et al, 1999), lack of glucose oxidase activity or hydrogen peroxide in NY buckwheat honey may well be compensated by phenolic constituents being present in relatively large amounts.

Two honeys, known as jelly bush and manuka honey are interesting because they can have anti-microbial activity, referred to as the non-peroxide activity (NPA), due to some property other than the production of hydrogen peroxide, low pH or high sugar content. Both jelly bush and manuka are plants that are Leptospermum species (Mwipatayi et al, 2004).

The advent of antibiotics in the 1950s revolutionized the control of bacterial infections, but with the recent escalating prevalence of bacterial resistance there has been renewed interest in the use of topical antimicrobials particularly silver, iodine, and honey (Leaper et al, 2004).

The therapeutic benefits of honey products have particular application in the field of chronic wound care, and particularly for infected wounds. Such honeys have been found to exhibit non-peroxide (antibacterial activity/phenol activity) as well as peroxide activity. Both of these activities are particularly advantageous in the care of wounds.

The major cause of the antibacterial activity is due to hydrogen peroxide that is produced in honey by the enzyme glucose oxidase. Manuka honey has been found to possess an amount of activity that is in addition to this antibacterial activity. This additional activity is known as the non-peroxide activity and is commercially known as unique manuka factor (UMF) (Willix et. al, (1992) (Cooper et al, 1999) (Cooper, R. A.; Molan, P. C., et al, 1999).

Chronic non healing wounds have an elevated alkaline environment. Lowering wound pH can potentially reduce protease activity, increase fibroblast activity and increase oxygen release consequently aiding wound healing. The use of Manuka honey dressings was associated with a statistically significant decrease in wound pH and a reduction in wound size (Gethin, 2008).

The pH of the wound can affect many factors including oxygen release, angiogenesis, protease activity, and bacterial toxicity. Chronic non-healing wounds have an elevated alkaline environment. Healing occurs more readily in an acid environment (Gethin, 2007).

The pH environment of chronic wounds has been recorded within the range of 7.15-8.9 (Wilson et al, 1979; Tsukada et al, 1992; Romanelli et al, 1997). Both acute and chronic wounds with an elevated alkaline pH have demonstrated lower rates of healing than wounds in which the pH is closer to neutral (Leveen et al, 1973; Roberts et al, 1997; Gethin and Cowman, 2006). Importantly, as the wound progresses towards healing, the pH moves to neutral and then becomes acidic (Tsukada et al, 1992; Kaufman et al, 1985). Indeed, the presence of necrotic tissue and devitalized tissue in the wound causes an increased metabolic load on the wound resulting in tissue hypoxia (Hunt and Beckert, 2005).

The pH environment also influences oxygen release to the tissues. Oxygen delivery to damaged tissue particularly in the chronic wound is dependent not only on perfusion but diffusion (Hunt and Beckert, 2005). A lowering of pH by 0.6 units releases almost 50% more oxygen and a five-fold increase in release of oxygen by a shift of 0.9 pH units (Leveen et al, 1973).

Indeed in chronic recurrent wounds such as venous leg ulcers, the skin and local vasculature become scarred and atrophic, resulting in permanent obstacles to the transport of oxygen (Hunt and Beckert, 2005). Therefore, any factor that could cause even a small change in the pH of the wound may appreciably alter the available supply of oxygen to the tissues (Leveen et al, 1973).

In addition to the effects on protease activity and oxygen release, other effects of lowering the pH to a more acidic environment are to: a. reduce the toxicity of bacterial end products such as ammonia, b enhancing the destruction of abnormal collagen in the ulcer bed, c. promotion of angiogenesis, d. increased macrophage and fibroblast activity and e. control of enzyme activity (Thomas, 1990; Romanelli et al, 1997; Molan, 2002; Brett, 2003; Greener et al, 2005).

Recent studies of this effect have included the use of honey dressings to alter surface pH (Gethin and Cowman, 2006). The surface pH and wound size of chronic non-healing wounds was monitored in 20 wounds over a two-week period (Gethin and Cowman, 2006). This study reported that wounds having a pH of ≦7.6 showed a 30% reduction in wound size after two weeks. As the pH increased, the reduction in size decreased. In addition, those with a pH of 8.0 or higher increased in size. The use of honey which has a pH of 3.5 showed a statistically significant reduction in surface wound pH after treatment (p=0.001) (Gethin and Cowman, 2006).

### Performing Autolytic Debridement

The formation of slough (dead tissue) on a wound is widely accepted as an inhibitor to natural wound healing. Diabetics, the elderly, and others with low mobility or poor circulation may encounter wounds that sometimes become chronic, and tends to collect this dead tissue or debris, called slough.

Cleansing of wound and removal of debris is a technique adopted very early in the history. Surgical debridement as a concept of wound care became a common empiric practice during the 18th century and was fully accepted in the 20th Century, when the research and the developments in microbiology were widely disseminated (Lindholm C, 2003).

At present times, debridement is a critical phase of “wound bed preparation”, which is considered the preliminary and fundamental process of any modern approach to the treatment of ulcers. It is increasingly required by the development of new products specifically dedicated to enhancing the healing of chronic ulcers. (Falanga, 2000) (Romanelli, 2002).

Autolytic debridement is the process by which the body attempts to shed devitalized tissue by the use of moisture. Where tissue can be kept moist this process is facilitated by the presence of enzymes (matrix metalloproteinase's) which break down protein bonds and lead to the sloughing away of non-viable tissue, (Thomas et al, 1999).

There are a large number of debridement options open to the practitioner, and the autolytic debridement can be divided into two categories: those that donate moisture to the dead tissue, and those that absorb excess moisture produced by the body. In the moisture donation category, product such as Hydrocolloids, hydrogels, honey and silver sulphadiazine donate moisture to the wound and thus enhance the process of debridement (Cooper et al, 2003).

Debridement, reduction of bioburden and exudates management is collectively referred to as Wound Bed Preparation. Wound Bed Preparation uses four principles in the acronym T.I.M.E which is based on intervention in four clinical areas and leads to an optimal, well vascularized wound bed.

In order to facilitate healing, slough is periodically removed. This procedure is called debriding the wound or debridement (U.S. Department of Health and Human Services, 1994). Honey appears to promote rapid wound debridement, replacement of sloughs with granulation tissue and rapid epithelialization and absorption of edema from around the margins.

Recent international medical literature record honey as being effective as a dressing for wounds, burns and skin ulcers. Recorded observations include that inflammation, swelling and pain are quickly reduced; that sloughing of necrotic tissue occurs without the need for debridement, and that growth of tissues to repair the wound is stimulated.

### Induction of Angiogenesis

Surgeons and wound-care specialists can use their knowledge of angiogenesis to identify defects and select interventions that may promote wound granulation because this process encourage the growth of new capillary and blood vessels. Clinically, new capillaries first become visible in the wound bed 3-5 days after injury, and their appearance is synonymous with granulation, the creation of a provisional matrix comprised of proliferating blood vessels, migrating fibroblasts and new collagen (Tonnesen et al, 2000).

The angiogenic process becomes active from day 2 after wounding (Grotendorst et al, 1984). Factors in the wound milieu that contribute to angiogenesis include high lactate levels, acidic pH, and, in particular, decreased oxygen tension (Witte et al, 1997) (Pencev et al, 1984). In this sense, by means of the formulation of the present invention, the PAD composition enhances angiogenesis, improves local circulation, and promotes stromal regeneration.

Defects in the angiogenesis process are present in diabetic foot ulcers, venous insufficiency ulcers, and arterial ulcers. Impaired circulation is an underlying pathological feature in peripheral arterial disease, ischemic heart disease, and chronic wounds (Li et al, 2003).

It is an object of the present invention to provide a wound dressing with which honey is used that addresses the above-mentioned problems, by the use of a substance mixture comprising honey, gelling agents, plant extracts and compounds from the group of Methyl-phenol and acetic acid for preparing a dressing for topical application.

The use of this information could help the clinician in making treatment decisions and ultimately a move towards a targeted therapeutic approach to wound management. Additional studies to explore this issue are required. Further aspects and advantages of the present invention will become apparent from the ensuing description which is given by way of example only.

### BRIEF SUMMARY OF THE INVENTION

The present invention includes a honey formulation that is suitable for topical application and/or to fill wound cavities into the warm-blooded vertebrate including human subjects. The formulation includes the combination of active ingredients of honey, gelling agents, plant extracts and compounds from the group of phenol and acetic acid. In the preparation the physical characteristics of honey are modified, while keeping the active ingredients intact for therapeutic purposes. Additionally the formulation, by the addition of selected components (group of Methyl-phenol and acetic acid constituents), synergistically enhancing the antimicrobial activity of honey that is beneficial to healing of wounds. Due to those healing mechanisms and antimicrobial activities, the present honey preparations are particularly effective in treatment of acute and chronic wounds, burns and ulcers of different etiologies. The present invention provides wound dressings which are easy to apply or fill wound cavities in very few steps for therapeutic purposes. These and further and other objects and features of the invention are apparent in the disclosure, which includes the above and ongoing written specification, with the claims.

### BRIEF DESCRIPTION OF THE SEVERAL VIEWS OF THE DRAWING

“Not Applicable”

### DETAILED DESCRIPTION OF THE INVENTION

### Description of the Preferred Embodiments of the Invention

### Definitions

In the discussions herein, a number of terms are used. In order to provide a clear and consistent understanding of the specification and claims, the following definitions are provided.

Acute—sharp, severe, having rapid onset, severe symptoms and a short course, not chronic

Angiogenesis—This occurs during the proliferative phase of healing when new blood vessels infiltrate the wound and endothelial budding forms capillaries. Adhering dressing—the quality of clinging or being closely attached

Antimicrobial agent—preventing the development or pathogenic action of microbes, helps decrease an infection and allows healing of a wound

Antiseptic—A substance that inhibits the growth and development of microorganisms without necessarily killing them. Antiseptics are usually applied to body surfaces.

Bacterial—unicellular microorganisms, lacking chlorophyll, many different kinds of bacteria, germs, creating an infection

Chronic—long, drawn out, applied to a disease that is not acute.

Chronic wound—covered with necrotic (dead) material and surrounded by cellulites, also is described as an infected wound.

Connective tissue—Refers to tissue that protects and supports the body and its organs, and tissues that bind organs together.

Debridement—the process of removing dead/unhealthy and/or contaminated tissues, foreign bodies, and other debris on the wound, using mechanical, enzymatic or sharp techniques.

Disinfectant—A chemical or mixture of chemicals used to kill microorganisms, usually applied to inanimate surfaces or objects.

Disinfection—A physical or chemical means of killing microorganisms, but not necessarily spores.

Dressing—Any of various materials utilized for covering and protecting a wound.

Edema—a condition in which the body tissues contain an excessive amount of tissue fluid. It may be local or general swelling

Enzymatic activity—an organic catalyst produced by living cells (as in digestive juices), but capable of acting independently of the cells producing them. They are capable of inducing chemical changes in other substances without themselves being changed in the process.

Epidermis—the outer layer of skin

Epithelialization—The process of the formation of new epithelial tissue—the upper layer of the skin—.

Eschar—a slough, especially one following a cauterization or burn—any agent used to destroy dead tissue and to cause sloughing which produces an eschar (a blackened area) in the treatment of skin disease

Excision—Is a surgical procedure requiring incision through the deep dermis (including subcutaneous and deeper tissues) of open wounds, burn eschar, or burn scars.

Exudates—the tan/grayish material that often forms over an open wound. It consists of proteinaceous material from the wound itself.

Granulation tissue—Newly formed vascular tissue (fragile capillaries) normally produced in the healing of wounds of soft tissue and ultimately forming the scar; it consists of small, translucent, pink to red, nodular masses of granulations that fill in an open wound bed during the proliferative phase of healing.

Infection—The host response to bacterial, viral or similar invasion. Inflammation—A non-specific host response to invasion of foreign material.

Lesion—any visible, local abnormality of the tissues of the body, as a wound, sore, rash, or a boil. A lesion may be described as benign, cancerous, gross, occult, or primary

Necrosis—localized tissue death that occurs in groups of cells in response to disease or injury, as an example—blood clots may block the flow of blood causing tissue ischemia below the blood clot, or ischemia combined with bacterial action can cause gangrene to set in Purulent—producing or containing pus

Ripe honey—It is meant honey naturally produced by bees to have water content, in a range known to those skilled in the art, which typically varies from about 14% to about 18% water by weight.

Sterilization—A process that kills and/or removes all classes of microorganisms and spores.

Stroma—The supportive framework of an organ usually composed of connective tissue.

Superficial—confined to the surface Transparent dressing—a dressing that you can see through and visualize the ulcer, but can be occlusive (taped on all four sides)

Tissue regeneration—Healing in which lost tissue is replaced by proliferation of cells which reconstruct the normal architecture.

Tissue repair—Healing in which lost tissue is replaced by a fibrous scar, which is produced from granulation tissue.

Treatment or treating—refers to any means of control of a condition or disorder, including prevention or prophylaxis, cure and relief, or relief of development of the condition or disorder.

Ulcer—a circumscribed, craterlike lesion of the skin or mucous membrane resulting from necrosis that accompanies some inflammatory, infectious, or malignant processes. An ulcer may be shallow, involving only the epidermis, or a deep crater going down to the bone

Wound dressing—is a material applied to a wound or a diseased part of the body, with or without medication, to protect and assist healing.

The invention is directed to a practical active dressing (PAD) honey-based composition, methods of producing the same, and therapeutic applications arising from their utilization. In one embodiment, the term “practical active dressing”, “(PAD)”, “dressing” or “wound dressing” refers to three-dimensional structure capable of covering-protecting a wound, performing autolytic debridement (moisture donation) and induction of angiogenesis.

In another embodiment, any reference to “including humans” placed after references to “warm blooded vertebrates” is intended to clarify that whilst humans are contemplated as recipients of the PAD composition. In another embodiment the ripe honey of bee for use in the (PAD) composition in accordance with the invention are typically prepared whit a preferred viscosity, a preferred pH, a preferred gelling agents, a preferred chemical compounds (for example antimicrobial) and preferred plant extracts.

In accordance whit one embodiment, the preparation of a PAD is carried out by the addition of preferred components (group of methyl-phenol and acetic acid constituents), and plant extract (Trigonella foenum-graecum), to the ripe honey, that synergistically enhancing the antimicrobial activity, performing autolytic debridement and the induction of angiogenesis that is beneficial to healing of wounds. The combination of these components results in the preparation of a PAD composition that is non-immunogenic, and, thus, does not induce an adverse host immune response when it is used to wound covering, for performing autolytic debridement (moisture donation) and induction of angiogenesis into a host. In another embodiment, this invention provides a process for preparing a practical active dressing (PAD) honey-based composition, and in general, the method for preparing the dressing comprises the steps of obtain ripe honey of bee from proper suppliers that ensure good manufacturing practices.

In a First Process the operator proceed to Reagent No. 1 preparation by mix Trigonella foenum-graecum seed, demineralized water and vinegar and let stand for three days. Then, in a Second Process the operator proceeds to mix the supernatant of reagent No. 1 with ripe honey, Agar Agar and the methil-phenil composition. After that, in a Third Process the operator proceeds to boil this composition until the time of starting the ebullition process. Next, in a Fourth Process proceed to distribution this mixed composition in and individual box and leave to dry in an environment free of contaminants Each box should be packaged individually into a vacuum bag (multi-layer synthetic bag) and the bag then is sealed. Afterward, in a Fifth process proceed to Eto Sterilization, which is carried in special chamber, for 24 hours and then air exposed for other 24 hours.

In another embodiment, the use of the practical active dressing (PAD) honey-based composition, in cases where native tissue is damaged, in one embodiment, by trauma, or in another embodiment, by burns, or in a further embodiment by vascular skin complications (chronic ulcers). In accordance with one embodiment, such practical active dressing compositions enables a surgeon to utilize this composition in a diverse range of surgical applications such as burns, wounds, vascular skin complications (chronic ulcers) or extensive injuries presented in theaters of war.

Thus, another embodiment of this invention is a method of implanting into the vertebrate the PAD composition, prepared as described above comprising the combination of active ingredients of ripe honey, gelling agents (Agar Agar), plant extracts (Trigonella foenum-graecum) and chemical compounds (group of Methyl-phenol and acetic acid) in an amount effective to reduce the pH for the regulation of bacterial growth, to performing Autolytic debridement and induce angiogenesis at the site of administration of the wound dressing. In another embodiment, it is thus the object of the present invention to provide an agent for topical application which has a broad application spectrum and thus may be employed for a plurality of uses, which are as close to natural as possible, and thus is well tolerable without any negative side effects.

In another embodiment, the PAD composition produced and used in accordance with this invention, upon implantation, can serve for the growth of new endogenous connective tissues in the warm-blooded vertebrate including humans. Connective tissues for the purposes of the present invention include the dermal layer of skin. In accordance with this embodiment, the PAD composition is used beneficially for wound debridement and induction of angiogenesis from the surrounding connective tissue at a desired site in a warm-blooded vertebrate including humans. In another embodiment, this invention provides a method of implanting a dressing of this invention in a subject for immediate wound cover, as show in example # 2 in this discovery.

In another embodiment, the practical active dressing is implanted in direct contact with the wound bed. In one embodiment, the dressing becomes sufficiently adhered to the wound bed, thoroughly integrated at all levels, and will performing wound debridement, induction of angiogenesis and preparation of the wound area for skin autograft, where such is considered additionally required. In another embodiment, is contemplated the use of the combination of active ingredients of ripe honey, gelling agents (Agar Agar), plant extracts (Trigonella foenum-graecum) and chemical compounds (group of Methyl-phenol and acetic acid) for the manufacture of other treatment methods or medical applications or products which have not been discussed in the present application, e.g., topical formulations (creams, ointments, gels), transdermal systems, microspheres for drug delivery system, or used for the purpose of improving, developing or enhancing other biotechnological/biological products.

As can be seen from the forgoing description, the concepts of the present disclosure provide numerous advantages The following examples are presented in order to more fully illustrate the preferred embodiments of the invention. They should in no way be construed, however, as limiting the broad scope of the invention.

### Pad Example 1

This invention provides a process for preparing a practical active dressing (PAD) honey-based composition, and in general, the method for preparing the dressing comprises the steps of obtain ripe honey of bee from proper suppliers that ensure good manufacturing practices In a First Process the operator proceed to Reagent No. 1 preparation by mix Trigonella foenum-graecum seed, demineralized water and vinegar and let stand for three days. Then, in a Second Process the operator proceeds to mix the supernatant of reagent No. 1 with ripe honey, Agar Agar and the methil-phenil composition. After that, in a Third Process the operator proceeds to boil this composition until the time of starting the ebullition process. Next, in a Fourth Process proceed to distribution this mixed composition in and individual box and leave to dry in an environment free of contaminants. Each box should be packaged individually into a vacuum bag (multi-layer synthetic bag) and the bag then is sealed. Afterward, in a Fifth process proceed to Eto Sterilization, which is carried in special chamber, for 24 hours and then air exposed for other 24 hours.

### PAD Example 2

Indications for use: The PAD honey-based composition may be used for temporary coverage of superficial and deep second degree burn (partial thickness burn) and for autolytic debridement and stimulated angiogenesis in full thickness wounds including necrotic or sloughy wounds, pressure ulcers, leg ulcers, surgical wounds, trauma wounds (abrasions, lacerations, erosions), donor sites and malodorous wounds.

Target patient group (s): patients with critically colonized wounds, patients with clinically infected wounds that are being treated with oral or intramuscular-intravenous antibiotic therapy.

### Preventive Aspect

Prior to the placement of the cover, it is necessary to perform several actions, due to the impact they have on the healing of wounds:

a. Acquiring a complete full medical history to obtain antecedents in relation to diseases such as: Diabetes, Vascular diseases, Connective tissue disorders, Allergies, Medication, Nutritional status, Lifestyle (Tobacco/alcohol habits, etc.) impaired mobility, and quality of life.

b. Assess the wound carefully recording the following: Location of wound, wound size (including cavity, sinus and fistula), characteristics of wound bed (necrosis, granulation, and infection), and presence of exudates (none, low, moderate, high), existence of odor (absent, present), clinical signs of local infection, abnormal granulation tissues or pain at wound site.

c. A bacterial culture of the deepest portion of the wound should be obtained when a patient is first seen if there is clinical evidence of infection, which is commonly polymicrobial.

d. Debridement and antibiotic therapy must be initiated as early as possible and the blood glucose should be monitored closely and controlled.

Additionally, following the initial wound assessment, the points below should be considered before starting the treatment: Complete evaluation of the patient (include comprehensive vascular evaluation). Determining the etiology of wound (i.e. acute, chronic, ischemic). Ascertain the underlying disease, e.g. venous/arterial. Use a validated pressure ulcer classification system to document the level of tissue loss (i.e. Braden Scale, Norton Scale, Waterlow Score Card, Braden Q (for pediatric patients), or other age appropriate tool in conjunction with clinical judgment. Utilize appropriate and regular measurement of wound size (take a tracing of the wounds). Determining the bacterial bioburden of the injury by tissue biopsy or quantitative swab technique (i.e. Levine quantitative swab technique). Assess for osteomyelitis or skin neoplasm's in chronic ulcers with appropriate Laboratory tests (biopsy of every non-healing wound) and x-rays as need. Consider a sharp debridement before application of the PAD composition. Nutritional assessment and lab evaluation of plasma proteins and Hematocrit-Hemoglobin. Such information it is essential to identify these factors and control them to facilitate faster wound healing whenever possible.

### (PAD) Placement Procedure for Temporary Covering

Prepare wounds very meticulous, to guarantee lesion is entirely excised or surgically debrided, to ensure the wound bed and edges contain viable structures, free of debris and necrotic tissue.

With normal saline solution gently irrigated the injury. Debride blister and loose skin. Cleanse with warmed normal saline. Avoid broad area of capillary bleeding which can lead to devitalized tissue. Prepare/open dressing items on table. Open the outer pocket used gloved hands. Place the product and nylon mesh in a sterile field. Translated onto the wound bed and gently remove the PAD composition with gloved hands. Cut to size with clean or sterile scissors and apply. The Dressing can overlap healthy tissue by 1 to 2 mm. If the wound is larger than a single sheet, then multiple sheets may be used.

To ensure close contact, make sure careful distribution, with no wrinkles or bubbles. Preparing Bowl with sterile saline solution and immersing the nylon mesh (second dressing) for 15 seconds. Next the tissue is overlaid with this material and bandaged with elastic tubular dressing (third dressing) to preserve adherence and permit easy inspections. Dressings Fixation and compression are of significant importance to ensure the PAD adheres to the wound without shear. In the Day, exposed the injury site to light (goose lamp 60 Watts) five minutes every two hours, for membrane desiccation for 48 hours. Reassess wounds after 24-48 hours and see the wound. Not touch the dressing until is advantageous to remove (seven or more days post implant). On inspection, at 48-72 hours, the PAD changes the yellow color to a brown aspect, similar to crust. Opaque and dry appearance indicates burn wound has re-epithelialized.

At seven or more days the PAD may gradually peel and may be removed with gently irrigation with saline solution or by shower. They ensured pain-free removal after complete healing of the wound. Do not forcibly remove sections of the PAD that may adhere to the wound. Later, with new irrigation, they remove completely. (PAD) placement procedure for autolytic debridement and stimulated angiogenesis: Prepare/open dressing items on table. Open the outer pocket used gloved hands.

Place the product and nylon mesh in a sterile field. Translated onto the wound bed, and gently remove the PAD composition of the container with gloved hands. Apply the PAD in direct contact with wound surface. If the wound is larger than a single graft, then multiple grafts may be used. Alternative to this method is the fragmentation of the graft and place inside a sterile syringe and the PAD composition in appearance of gel can be applied also to deep wounds directly. This safe application is promoted by the gel consistency, utilized to fill the wound. The gel is sufficiently packed to prevent immediate escape and soft sufficiently to adapt to the wound base. Pack it lightly with nylon net and cover the packed wound with a dry elastic bandage.

### Dressings to Protect the Practical Active Dressing as Follows

High-quality white nylon net and a Tubular Elastic Bandage applied over nylon layer, to protect the site and to reduce the potential of shearing and graft dislodgement. Both can be left in place for extended periods without detrimental effects to the underlying wound. In this sense, preparing Bowl with sterile saline solution and immersing the nylon mesh (second dressing) for 10 seconds.

Next the wound bed is overlaid with this material in direct contact with wound surface and then bandaged with elastic tubular dressing (third dressing) to preserve adherence and permit easy inspections. Take care in the selection of the appropriate stretch of the tubular dressing in arterial leg ulceration, because they impaired circulation in this kind of patients.

Complications: The following complications are possible with the use of any wound dressings. If any of the conditions occur, the PAD composition should be removed: infection, inflammation, allergic reaction, excessive redness, pain or swelling. Complications that the surgeon may encounter further than infection include seroma and/or hematoma formation, and graft contracture. Although wound infection is rare, when this is suspected, appropriate bacterial identification is obligatory and according to the cultivated microorganisms antibiotics should be prescribed.

### PAD Example 3

It is presented in this example the result of an open, prospective, comparative, controlled trial with random distribution of cases, conducted at the Department of Pediatric Surgery in the Public General Hospital “San Juan de Dios”, of the Government of Guatemala, in the period from August 1985 to January 1986.

This clinical research refers to an interventional study, to treat patients in a single center, in order to examine treatment variation and patient outcomes, including the effect of new management strategies, implementation of practice guidelines, and quality improvement initiatives.

The use of sulfadiazine Argentic cream (SS), and the Practical Active Dressing (B) were compared in the management of partial thickness burns. 18 children were studied, with less than 10% of body surface burned.

It should be emphasized that many patients with minor burns were hospitalized derived from different social circumstances, values, beliefs, health care practices, cultural barriers or some issues: such as lack of transport for dressing changes, and additionally by the high incidence of non-accidental burns and scalds, in children with suspected mistreatment.

It should also highlight two important aspects: the poor nutritional status of these patients and the delay in attending the hospital in the first moments of the accident, with the use of home remedies during the initial days of the injuries.

### Methods

The Institutional Review Board (IRB) approved protocol and informed consent document for the study before inclusion, and authorization was requested in writing of the parents of minors.

Assessment of burn size: by use the Lund and Browder chart for % Total Body Surface Area. The depth of a burn injury should be reassessed two to three days after the initial assessment, by the same clinician.

### Types of Outcome Measures

Primary outcomes: time to complete wound healing and proportion of participants with completely healed wounds.

Secondary outcomes: Incidence of adverse events; Hospital length of stay; Change in wound size or deeper conversion; Incidence of infection; and Cost.

Treatment protocols stipulate low starting doses of analgesia and allow for adjustments to be made based on individual pain assessment.

### Application Technique

The burn team accomplishes all wound management. Initial treatment of the burn wound involves cleansing the wound and removes dirt and debris without damaging the burn wound. Ruptured blisters are removed with scissors. After wound cleansing, cover with a dressing or cream according to the previously established random selection.

The treatment site was checked daily by the same team of surgeons, to observe the progress until complete epithelialization and infection suspicious lesion was cultivated for determining pathogens.

### Results

Of the 18 cases (100%) eleven were male (61%) and seven female (39%).

61% of patients (11 cases) were between the ages of 0-5 years.

In both groups (SS) and (B), 8 patients for each group were treated with burns from scalds and one patient for each group of burns from flame.

In the most significant results included: average hospital stay of 27 days of the first method (SS) versus 12 days of the second (B).

54 wound manipulations need per patient for complete epithelialization, in the first group (SS) against three in the second (B).

Deepening to more complex lesion in 77% of cases of the first method (SS) against 11% of the second (B).

### Analysis

It is emphasized that a greater number of cures the possibilities of deepening the initial injury are extremely high, which contributes to leave permanent scars on the site of the injury.

Additionally the extension of days of hospitalization required, necessary care, cost, etc.

1. A practical active dressing (PAD) honey-based composition capable of covering-protecting a wound, performing autolytic debridement (moisture donation) and induces angiogenesis, when is positioned in warm-blooded vertebrate including human subjects. It comprises the combination of active ingredients of ripe honey, gelling agents (Agar Agar), plant extracts (Trigonella foenum-graecum) and chemical compounds (group of Methyl-phenol and acetic acid).

2. The method for preparing a honey-based composition to render it useful for the purposes described herein.

3. The honey-based composition will have a preferred viscosity, a preferred pH, a preferred gelling agent, a preferred chemical compounds (for example antimicrobial) and preferred plant extracts.

4. The honey-based composition of claim 3, in which the preferred gelling agent is Agar Agar, in which the preferred chemical agent by means of pH reduction is Policresulen (Condensation product of metacresolsulfonic acid & methanal), in which the preferred plant extract is from Trigonella foenum-graecum seeds.

5. The honey-based composition of claim 1, wherein the practical active dressing (PAD) is positioned by surgical means into the warm-blooded vertebrate including human subjects, according of the second example in the present invention.

6. The method of claim 5, wherein said method is used in the treatment of skin loss or damage caused by any type of injury or disease or surgical intervention conducted according to the methods of this invention.

7. A method for inducing the formation of endogenous tissue at a site in need of endogenous tissue growth in a warm blooded vertebrate including human subjects, said method consists of contacting said site with a practical active dressing (PAD) comprising the combination of active ingredients of ripe honey, gelling agents (Agar Agar), plant extracts (Trigonella foenum-graecum) and chemical compounds (group of Methyl-phenol and acetic acid).

8. The use of honey-based composition of claim 1 in the manufacture of other treatment methods or medical applications which have not been discussed in the present application, e.g., topical formulations (creams, ointments, gels), transdermal systems, microspheres for drug delivery system, or used for the purpose of improving, developing or enhancing other biotechnological/biological products.
